(19) United States
(12) Patent Application Publication
(10) Pub. No.: US 2010/0196300 A1
(43) Pub. Date: Aug. 5, 2010

(54) BASIC ALUMINUM NITRATE

(76) Inventor: Demetrius Michos, Clarksville, MD (US)

Correspondence Address: W.R. GRACE & CO.-CONN., 7500 GRACE DRIVE, COLUMBIA, MD, 21044, US

(21) Appl. No.: 12/669,229

(22) PCT Filed: Jul. 17, 2008
(86) PCT No.: PCT/US08/08770

§ 371 Date: Jan. 15, 2010

Related U.S. Application Data
(60) Provisional application No. 60/959,872, filed on Jul. 17, 2007.

(51) Int. Cl. C04B 35/111; A61K 8/26; A61Q 15/00; C04B 14/00

(52) U.S. Cl. 424/68; 501/153; 106/401

(57) ABSTRACT

A basic aluminum composition comprising an empirical formula: Al₂(OH)₆—ₐXₐ where 0.5≦a≦5.0, and X is an anion of nitrogen; and wherein the composition possesses an NMR spectrum in which a −40 to +40 ppm resonance line comprises at least 60% of the total area of the NMR spectrum.

### BACKGROUND OF THE INVENTION

The present invention relates to a basic aluminum nitrate composition and a method of making such basic aluminum nitrate.

Basic aluminum nitrate compositions are suitable for use in a variety of applications, such as a surface modifier, a binder, a ceramic, an antiperspirant, etc. Aqueous solutions of basic aluminum nitrate (aluminum oxynitrate) may be produced by dissolving a compound of aluminum, for example aluminum hydroxide or alumina, in nitric acid or aqueous aluminum nitrate solutions.

U.S. Pat. No. 5,202,115, provides for the production of basic aluminum materials that are utilized in antiperspirant formulations. The reaction scheme for forming the basic aluminum material is described as follows:

58H₂O+28Al+16Al(NO₃)₃→22Al₂(OH)₅NO₃+26NO+3H₂,

where the production of nitric oxide represents a reduction of the nitrate oxoanion from a formal +5 to +2 oxidation state on the nitrogen atom. This patent also discloses that the aluminum salt of the univalent complex oxoanion can be formed in situ, by reacting aluminum metal with, e.g., an inorganic acid of the univalent complex oxoanion. Consistent with techniques in connection with conventional basic aluminum halide materials, this patent describes forming the described basic aluminum material having the univalent complex oxoanion by adding small turnings of aluminum metal in the form of oblong pieces 1/16 inch to ⅛ inch long and 1/100 inch to 3/100 inch thick, in excess, to a solution of monomeric aluminum ion and univalent complex oxoanion.

U.K. Patent Application No. 2,048,229 describes a group of complexes (Al^{Cr}) within the aluminum chlorhydroxides, which are more efficacious as an antiperspirant. Such group Al^{Cr} complexes with a ferron reagent at a reaction rate characteristic of Al^{c} (of Al^{a}, Al^{b} and Al^{c}, Al^{c} is the group that exhibits the slowest complexing reaction ratio with ferron), and has a permeation rate in gel permeation chromatography which is within that range generally found for Al^{b} (of Al^{a}, Al^{b} and Al^{c}, Al^{b} has an intermediate retention time, indicating it includes complexes of intermediate molecular size). This U.K. patent application describes that the Al^{Cr} group of complexes was present in amounts of 10%-30% by weight in then-available aluminum chlorhydroxides, and that these then-available aluminum chlorhydroxides can be modified to contain substantially lager amounts of the Al^{Cr} group. This patent application discloses a technique to increase the amount of the Al^{Cr} group, by aging aluminum chlorhydroxide.

European Patent Application No. 191,628 discloses a direct process of making a basic aluminum halide in powder form having an aluminum:halogen molar ratio of from 1.7 to 2.2:1. This process includes steps of: (a) dissolving metallic aluminum, in an aqueous starting solution of an aluminum compound selected from aluminum chloride and aluminum bromide, the starting solution being held at a temperature of about 50° C. to about 105° C., for a time just long enough to dissolve sufficient aluminum to produce an aqueous solution of a final basic aluminum halide having an aluminum:halide molar ratio in the range 1.7:1 to 2.2:1, the concentration of the aluminum in the starting solution and the amount of aluminum dissolved being such that the aluminum concentration in the solution of the final basic aluminum halide is from 0.8% to 6.75% by weight; and (b) drying the solution of the final basic aluminum halide.

European Patent Application No. 191,628 describes a direct preparative procedure for forming the described basic aluminum halide material containing a high proportion of the aluminum in the form of a polymer having a characteristic line in the ²⁷Al NMR (nuclear magnetic resonance) spectrum. This patent application discloses this characteristic line is 62.5 ppm downfield from the resonance of Al³⁺(6H₂O), and has been attributed to a complex aluminum ion referred to as the Al₁₃O₄₀ ion. In one embodiment of the disclosed process, at least 20% of the aluminum of the final basic aluminum compound is in the form of the Al₁₃O₄₀ ion.

European Patent Application No. 285,282 discloses antiperspirant materials, including partially neutralized aluminum salts, the salts having at least 25% of the total aluminum present in a form having a ²⁷Al NMR spectrum wherein 8% to 25% of the total area under the spectrum from 140 ppm to −80 ppm is contained in a peak at approximately 63 ppm (corresponding to tetrahedrally coordinated aluminum ions). This European patent document describes a technique for forming the described aluminum salt by partially neutralizing an aqueous acid (such as a mineral acid) using a source of aluminate ion (the mineral acid optionally being an aluminum salt), with no subsequent heating step required. Specifically embodied in this patent document are aluminum halohydrate materials, such as aluminum chlorhydrate.

U.S. Pat. No. 5,626,827 describes basic aluminum materials (polymeric aluminum materials) having certain size exclusion high performance liquid chromatography peaks produced from a high performance liquid chromatography (HPLC) with less than 25% of the aluminum being in the form of Al^{b} polyhydroxyaquoaluminum; and an ²⁷Al NMR (nuclear magnetic resonance) spectrum wherein 5%-30%, preferably 8%-18%, of the total area under the spectrum from 140 ppm to −80 ppm is contained in a resonance line at 71.5-73.5 ppm; and an ²⁷Al NMR spectrum in which the area of the 71.5-73.5 ppm resonance line includes more than 50% of the combined areas of the 62.5-63.5 ppm and 71.5-73.5 ppm resonance lines.

U.K. Patent Application No. 2,053,172 A describes a process for the preparation of a stable, liquid aqueous solution of basic aluminum nitrate (aluminum oxynitrate) which is suitable for spinning into fibres, if desired after incorporation of a polymeric spinning aid. The process comprises (i) reacting an aluminum oxide with nitric acid solution at a temperature below 25° C., to form aluminum hydroxide, (ii) separating the resulting aluminum hydroxide precipitate, (iii) if necessary removing alkali metal ions and other undesirable ions from the precipitate, and (iv) digesting the aluminum hydroxide precipitate in nitric acid or aluminium nitrate.

However, it has not been possible hitherto to produce satisfactory stable solutions having an acceptable aluminum to nitrate molar ratio and also maintain high purity.

### SUMMARY OF THE INVENTION

In one embodiment, the present invention relates to a basic aluminum composition having an empirical formula:

Al₂(OH)₆₋ₐXₐ

where 0.5≦a≦5.0, and X is an anion of nitrogen; and wherein said composition possesses an NMR spectrum in which a −40 to +40 ppm resonance line comprises at least 60% of the total area of the NMR spectrum.

In another embodiment, the present invention relates to a basic aluminum composition having an empirical formula:

Al₂(OH)₆₋ₐXₐ

where 0.5≦a≦5.0, and X is an anion of nitrogen; and wherein the composition comprises less than 3% by weight metal oxide impurities based on the total alumina weight of the composition.

In an even further embodiment, the present invention relates to a basic aluminum composition having an empirical formula:

Al₂(OH)₆₋ₐXₐ

where 0.5≦a≦5.0, and X is an anion of nitrogen; and wherein the composition possesses an NMR spectrum in which substantially no other resonance line other than at least one resonance line within −40 to +40 ppm is present in the NMR spectrum.

In a further embodiment, the present invention relates to a method of preparing a basic aluminum nitrate composition comprising, reacting aluminum oxide metal salt with nitric acid at a pH of less than or equal to about 6 to form alumina precipitate and metal nitrate; removing said metal nitrate from the precipitate; adding nitric acid to the precipitate to form a slurry and to adjust aluminum to nitrate ratio of the precipitate; and heating the slurry to form a solution of the basic aluminum nitrate.

### BRIEF DESCRIPTION OF THE DRAWINGS

FIG. 1 is a NMR spectrum for basic aluminum nitrate composition of the prior art.

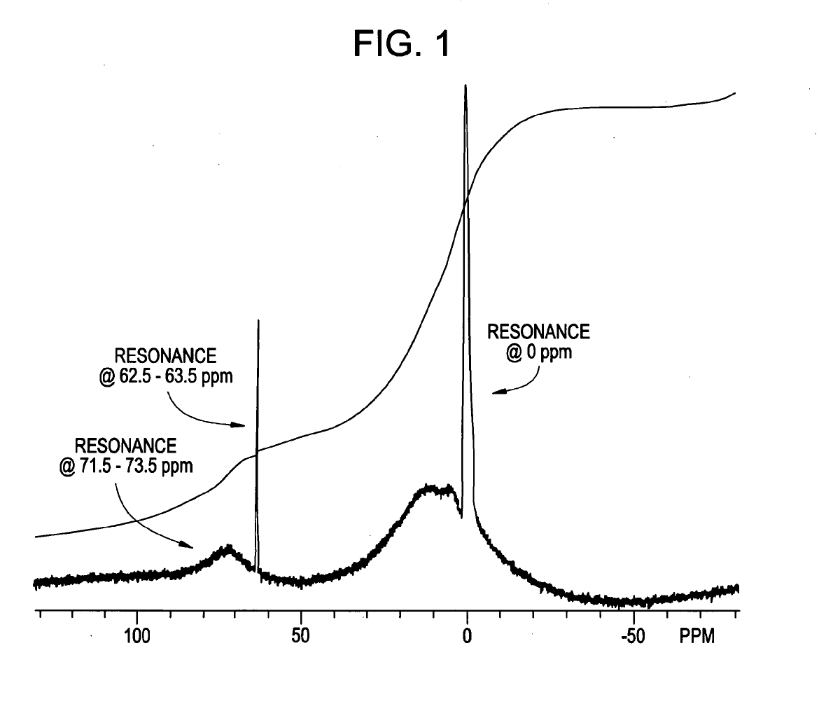

FIG. 2 is a NMR spectrum for basic aluminum nitrate composition of the prior art.

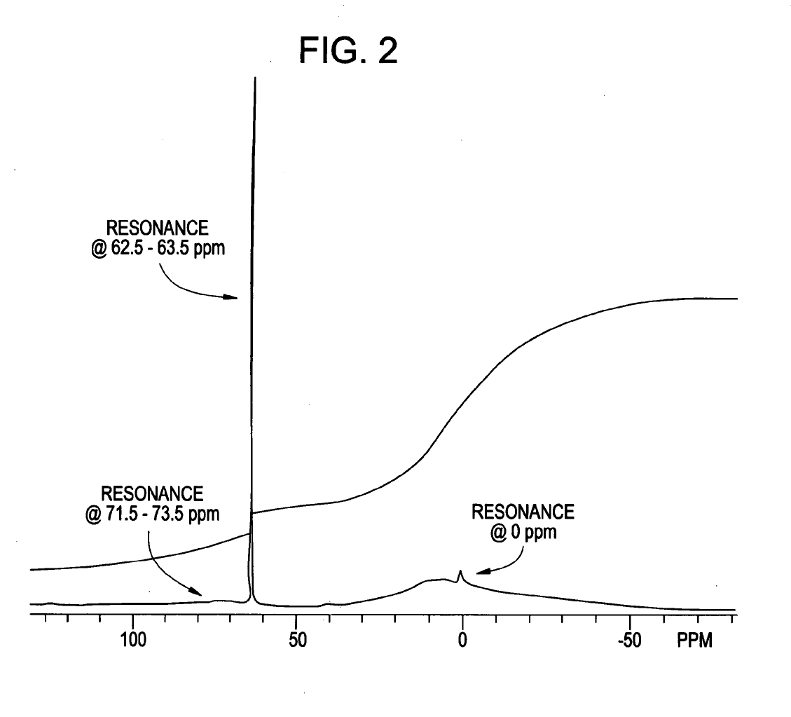

FIG. 3 is a NMR spectrum for basic aluminum nitrate composition according to the present invention.

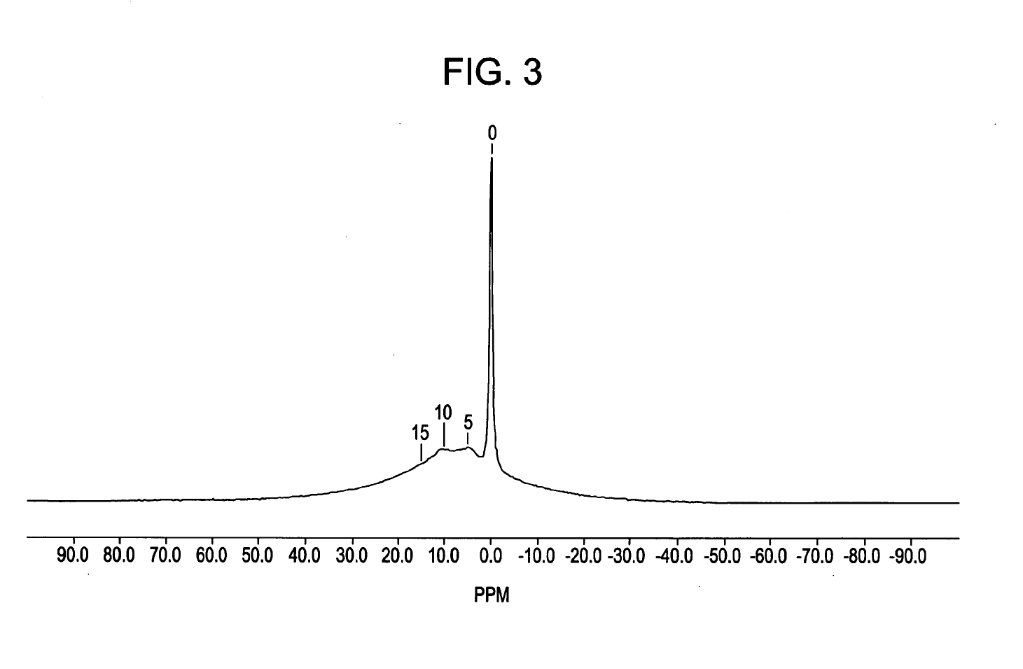

### DETAILED DESCRIPTION OF THE INVENTION

The present invention relates to a basic aluminum nitrate composition and a method of making such basic aluminum nitrate.

It must be noted that as used herein and in the appended claims, the singular forms “a”, “and”, and “the” include plural referents unless the context clearly dictates otherwise. Thus, for example, reference to “a basic aluminum nitrate composition” includes a plurality of such compositions and reference to “basic aluminum nitrate composition” includes reference to one or more such compositions and equivalents thereof known to those skilled in the art, and so forth.

“About” modifying, for example, the quantity of an ingredient in a composition, concentrations, volumes, process temperatures, process times, recoveries or yields, flow rates, and like values, and ranges thereof, employed in describing the embodiments of the disclosure, refers to variation in the numerical quantity that can occur, for example, through typical measuring and handling procedures; through inadvertent error in these procedures; through differences in the ingredients used to carry out the methods; and like proximate considerations. The term “about” also encompasses amounts that differ due to aging of a formulation with a particular initial concentration or mixture, and amounts that differ due to mixing or processing a formulation with a particular initial concentration or mixture. Whether modified by the term “about” the claims appended hereto include equivalents to these quantities.

As described herein, the term “basic” means a compound that is more alkaline than other compounds of the same name. For example, in the chemical formula mentioned herein, if a<6 then the compound is “basic”. For a=6 then the compound may be represented by Al₂(NO₃)₆ or Al(NO₃)₃ which is neutral aluminum nitrate with mole ratio Al/NO₃=0.333.

As utilized herein, the term “impurities” means anything other than, H, O, N, Al (in element form) or H₂O, NO₃ and the Al₂(OH)₆₋ₐXₐ. Impurities include, for example, metals and non-metals and any derivatives thereof, such as metal oxides (e.g., Na₂O, Fe₂O₃, MgO, TiO₂, ZrO₂, CaO, etc.), halides, sulfates and other oxoanions, and mixtures thereof.

As used herein, the term “metal oxides” means a compound that contains a metal cation and an oxide anion that typically reacts with water to form bases or with acids to form salts. Metals typically fall into the following classifications, but are not mutually exclusive and not rigidly defined: alkali metals, alkaline earth metals, transition elements, noble (precious) metals, platinum metals, lanthanide (rare earth) metals, actinide metals, light metals and heavy metals, and mixtures thereof.

In one embodiment, the present invention relates to a basic aluminum composition comprising an empirical formula:

Al₂(OH)₆₋ₐXₐ

where 0.5≦a≦5.0, and X is an anion of nitrogen; and wherein the composition possesses an NMR spectrum in which a −40 to +40 ppm resonance line comprises at least about 60% of the total area of the NMR spectrum, or the −40 to +40 ppm resonance line comprises at least about 70% of the total area of the NMR spectrum, or the −40 to +40 ppm resonance line comprises at least about 80% of the total area of the NMR spectrum, or the −40 to +40 ppm resonance line comprises at least about 90% of the total area of the NMR spectrum, or the −40 to +40 ppm resonance line comprises at least about 95% of the total area of the NMR spectrum. In another embodiment, the NMR spectrum comprises substantially no resonance line other than at least one resonance line appearing within −40 to +40 ppm of the spectrum.

In an embodiment, the composition may comprise less than about 3% by weight metal oxide impurities based on the total alumina (expressed as Al₂O₃) weight of said composition, or less than about 1% by weight metal oxide impurities based on the total alumina weight of said composition, or less than about 0.5% by weight metal oxide impurities based on the total alumina weight of said composition, or less than about 0.1% by weight metal oxide impurities based on the total alumina weight of said composition, or less than about 0.07% by weight metal oxide impurities based on the total alumina weight of said composition. The metal oxide impurities may include, but are not limited to, oxides of sodium, iron, magnesium, titanium, zirconium, calcium or mixtures thereof. The composition may comprise less than about 0.3% by weight sodium oxide impurities based on the total alumina weight of said composition, or less than about 0.07% by weight sodium oxide impurities based on the total alumina weight of said composition.

In another embodiment, present invention relates to a basic aluminum composition comprising an empirical formula:

Al₂(OH)₆₋ₐXₐ

where 0.5≦a≦5.0, and X is an anion of nitrogen; and wherein the composition comprises less than about 3% by weight metal oxide impurities based on the total alumina weight of the composition, or less than about 1% by weight metal oxide impurities based on the total alumina weight of said composition, or less than about 0.1% by weight metal oxide impurities based on the total alumina weight of said composition, or less than about 0.07% by weight metal oxide impurities based on the total alumina weight of said composition. The metal oxide impurities may include, but are not limited to, oxides of sodium, iron, magnesium, titanium, zirconium, calcium or mixtures thereof. The composition may comprise less than about 0.3% by weight sodium oxide impurities based on the total alumina weight of said composition, or less than about 0.07% by weight sodium oxide impurities based on the total alumina weight of said composition. The composition possesses an NMR spectrum in which a −40 to +40 ppm resonance line comprises at least about 60% of the total area of the NMR spectrum, or the −40 to +40 ppm resonance line comprises at least about 70% of the total area of the NMR spectrum, or the −40 to +40 ppm resonance line comprises at least about 80% of the total area of the NMR spectrum, or the −40 to +40 ppm resonance line comprises at least about 90% of the total area of the NMR spectrum, or the −40 to +40 ppm resonance line comprises at least about 95% of the total area of the NMR spectrum. In another embodiment, the NMR spectrum comprises substantially no resonance line other than at least one resonance line within −40 to +40 ppm of the spectrum.

In a further embodiment, the present invention relates to a method of preparing a basic aluminum nitrate composition including, reacting aluminum oxide metal salt with nitric acid at a pH of less than or equal to about 6 to form alumina precipitate and metal nitrate; removing the metal nitrate from the precipitate; adding nitric acid to the precipitate to form a slurry and to adjust aluminum to nitrate ratio of the precipitate; and heating the slurry to form a solution of the basic aluminum nitrate. The metal may include sodium, potassium, or mixtures thereof. The aluminum oxide metal salt may include sodium aluminate, potassium aluminate or mixtures thereof. The metal nitrate may include sodium nitrate, potassium nitrate, or mixtures thereof. The aluminum oxide metal salt may be reacted with the nitric acid at a pH of less than or equal to about 5.5, or less than or equal to about 5.0, or less than or equal to about 4.5, or less than or equal to about 4. The nitric acid is added in an amount that is necessary to maintain the desired pH. The metal nitrate may be removed from the precipitate by washing with deionized water using ultrafiltration, or by other suitable means. After washing, nitric acid is added to the precipitate to form a solution such that the aluminum to nitrate mole ratio of the precipitate may be equal to or greater than about 1.0, equal to or greater than about 1.2, or equal to or greater than 1.4. The solution may be concentrated by removal of water using conventional techniques such as by distillation, evaporation, centrifuge or similar technique. The solution may also be filtered any time after formation to remove impurities such as by depth filtration or the like. If desired, the solution may further be dried to form a powder of basic aluminum nitrate such as, for example, by spray drying, tray drying or similar method.

### EXAMPLES

The following Examples are given as specific illustrations of the claimed invention. It should be understood, however, that the invention is not limited to the specific details set forth in the Examples.

### Example 1

3200 g of room temperature deionized water are charged in an 18 liter reactor, equipped with baffles and a stirrer. Sodium aluminate (4186 g; 11.5% Al₂O₃) is added simultaneously with a 20% nitric acid solution (4928 g) in order to maintain a pH of ˜4 for 20 minutes to form precipitated alumina and sodium nitrate. The final temperature is 41.6° C. 3078 g of the resulting alumina is filtered and washed using a filter funnel with 9.2 Kg of deionized water to remove the sodium nitrate byproduct. This filtration is repeated three more times, in order to wash the entire contents of the reactor. The resulting filter cake, 3390 g, has an Al/NO3 mole ratio of 5.6. The filter cake is then liquefied with the application of shear, using a dispersion blade. To that, 656 g of 40% nitric acid is added in order to adjust the mole ratio of Al/NO3 to ˜1.5. The resulting slurry is heated to 95° C. for approximately 2 hours in order to obtain the desired NMR spectrum. The resulting solution is analyzed and found to contain 17.7% Al₂O₃ and less than 20 ppm of Na₂O. The solution is ready to use and is remains stable for at least six months. The empirical formula of the basic aluminum composition is Al₂(OH)_{4.7}(NO₃)_{1.3}.

### Example 2

2100 g of room temperature deionized water are charged in an 18 liter reactor, equipped with baffles and a stirrer. Aluminum sulfate (8050 g; 8.3% Al₂O₃) is added simultaneously with a 25% sodium carbonate solution (9289 g) in order to maintain a pH of ˜5.3 for 20 minutes to form precipitated alumina and sodium sulfate. The alumina slurry is filtered and washed using a filter funnel with six volumes of deionized water to remove the sodium sulfate byproduct. The resulting filter cake, 4980 g, is dispersed in a minimum amount of deionized water using a dispersion blade. To that 1362 g of 40% nitric acid is added in order to adjust the mole ratio of Al/NO3 to ˜1.5. The resulting slurry is heated to 95° C. for approximately 2 hours in order to obtain the desired NMR spectrum. The resulting solution is analyzed and found to contain 16.3% Al₂O₃ , 104 ppm of Na₂O, 39 ppm of Fe₂O₃ and 12 ppm of MgO. The solution is ready to use and is remains stable for at least six months. The empirical formula of the basic aluminum composition is Al₂(OH)_{4.7}(NO₃)_{1.3}.

While the invention has been described with a limited number of embodiments, these specific embodiments are not intended to limit the scope of the invention as otherwise described and claimed herein. It may be evident to those of ordinary skill in the art upon review of the exemplary embodiments herein that further modifications, equivalents, and variations are possible. All parts and percentages in the examples, as well as in the remainder of the specification, are by weight unless otherwise specified. Further, any range of numbers recited in the specification or claims, such as that representing a particular set of properties, units of measure, conditions, physical states or percentages, is intended to literally incorporate expressly herein by reference or otherwise, any number falling within such range, including any subset of numbers within any range so recited. For example, whenever a numerical range with a lower limit, R_{L}, and an upper limit R_{U}, is disclosed, any number R falling within the range is specifically disclosed. In particular, the following numbers R within the range are specifically disclosed: R=R_{L}+k(R_{U}−R_{L}), where k is a variable ranging from 1% to 100% with a 1% increment, e.g., k is 1%, 2%, 3%, 4%, 5% . . . 50%, 51%, 52%. . . . 95%, 96%, 97%, 98%, 99%, or 100%. Moreover, any numerical range represented by any two values of R, as calculated above is also specifically disclosed. Any modifications of the invention, in addition to those shown and described herein, will become apparent to those skilled in the art from the foregoing description and accompanying drawings. Such modifications are intended to fall within the scope of the appended claims. All publications cited herein are incorporated by reference in their entirety.

What is claimed is:

1. A basic aluminum composition comprising an empirical formula:
Al₂(OH)₆₋ₐXₐ
where 0.5≦a≦5.0, and X is an anion of nitrogen; and wherein said composition possesses an NMR spectrum in which a −40 to +40 ppm resonance line comprises at least 60% of the total area of the NMR spectrum.

2. A composition according to claim 1, wherein said −40 to +40 ppm resonance line comprises at least 70% of the total area of the NMR spectrum.

3. A composition according to claim 1, wherein said −40 to +40 ppm resonance line comprises at least 80% of the total area of the NMR spectrum.

4. A composition according to claim 1, wherein said −40 to ₊40 ppm resonance line comprises at least 90% of the total area of the NMR spectrum.

5. A composition according to claim 1, wherein said −40 to +40 ppm resonance line comprises at least 95% of the total area of the NMR spectrum.

6. A composition according to claim 1, wherein said NMR spectrum comprises substantially no other resonance line other than at least one resonance line within −40 to +40 ppm present in said NMR spectrum.

7. A composition according to claim 1, wherein said composition comprises less than 3% by weight metal oxide impurities based on the total alumina weight of said composition.

8. A composition according to claim 1, wherein said composition comprises less than 1% by weight metal oxide impurities based on the total alumina weight of said composition.

9. A composition according to claim 1, wherein said composition comprises less than 0.1% by weight metal oxide impurities based on the total alumina weight of said composition.

10. A composition according to claim 8, wherein said metal oxide impurities comprises oxides of sodium, iron, magnesium, titanium, zirconium, calcium or mixtures thereof.

11. A composition according to claim 8, wherein said composition comprises less than 0.2% by weight sodium oxide impurities based on the total alumina weight of said composition.

12. A basic aluminum composition comprising an empirical formula:
Al₂(OH)₆₋ₐXₐ
where 0.5≦a≦5.0, and X is an anion of nitrogen; and wherein said composition comprises less than 3% by weight metal oxide impurities based on the total alumina weight of said composition.

13. A composition according to claim 12, wherein said composition comprises less than 1% by weight metal oxide impurities based on the total alumina weight of said composition.

14. A composition according to claim 12, wherein said composition comprises less than 0.1% by weight metal oxide impurities based on the total alumina weight of said composition.

15. A composition according to claim 12, wherein said metal oxide impurities comprise oxides of sodium, iron, magnesium, titanium, zirconium, calcium or mixtures thereof.

16. A composition according to claim 12, wherein said composition comprises less than 0.2% by weight sodium oxide impurities based on the total alumina weight of said composition.

17. A composition according to claim 12, wherein said composition possesses an NMR spectrum in which a −40 to +40 ppm resonance line comprises at least 60% of the total area of the NMR spectrum.

18. A composition according to claim 12, wherein said −40 to +40 ppm resonance line comprises at least 70% of the total area of the NMR spectrum.

19. A composition according to claim 12, wherein said NMR spectrum comprises substantially no other resonance line other than at least one resonance line within −40 to +40 ppm present in said NMR spectrum.

20. A basic aluminum composition comprising an empirical formula:
Al₂(OH)₆₋ₐXₐ
where 0.5≦a≦5.0, and X is an anion of nitrogen; and wherein said composition possesses an NMR spectrum in which substantially no other resonance line other than at least one resonance line within −40 to +40 ppm is present in said NMR spectrum.

21. A method of preparing a basic aluminum nitrate composition comprising,
reacting aluminum oxide metal salt with nitric acid at a pH of less than or equal to about 6 to form alumina precipitate and metal nitrate;
removing said metal nitrate from said precipitate;
adding nitric acid to said precipitate to form a slurry and to adjust aluminum to nitrate ratio of said precipitate; and
heating said slurry to form a solution of said basic aluminum nitrate.

22. A method according to claim 21, wherein said metal is sodium, potassium, or, mixtures thereof.

23. A method according to claim 21, wherein said aluminum oxide metal salt is sodium aluminate.

24. A method according to claim 21, wherein said metal nitrate is sodium nitrate.

25. A method according to claim 21, wherein said pH is less than or equal to about 5.

26. A method according to claim 21, wherein said pH is less than or equal to about 4.5.

27. A method according to claim 21, wherein said metal nitrate is removed by washing with deionized water.

28. A method according to claim 21, wherein said aluminum to nitrate ratio is equal to or greater than about 1.0.

29. A method according to claim 21, wherein said aluminum to nitrate ratio is equal to or greater than about 1.2.

30. A method according to claim 21, wherein said solution is concentrated by removal of water.

31. A method according to claim 21, wherein said solution is filtered to remove impurities.

32. A method according to claim 21, wherein said solution is dried to form a powder of said basic aluminum nitrate.
